(19) United States
(12) Patent Application Publication
(10) Pub. No.: US 2022/0096878 A1
(43) Pub. Date: Mar. 31, 2022

(54) AIR FILTER

(71) Applicant: Progress Luv2Pak International Ltd., Downsview (CA)

(72) Inventor: David Irwin Hertzman, Downsview (CA)

(73) Assignee: Progress Luv2Pak International Ltd., Downsview (CA)

(21) Appl. No.: 17/039,960

(22) Filed: Sep. 30, 2020

(51) Int. Cl. A62B 23/02; B01D 39/16

(52) U.S. Cl. CPC A62B 23/02; B01D 39/16; B01D 2239/0478; B01D 2239/1291; B01D 2239/0622; B01D 2239/0627; B01D 2239/0618

(57) ABSTRACT

The present invention relates to an air filter, and more particularly to a multilayer air filter incorporating nanosilver.

### FIELD OF THE INVENTION

The present specification relates generally to an air filter, and more particularly to a multilayer air filter incorporating nanosilver.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

Air filters are ubiquitous in daily life. Air filter are integrated into residential and commercial HVAC systems, personal and commercial vehicle air system, and specialized equipment such as CPAP machines. Recent air transmissible pandemic events (SARS, COVID) have also increased usage in non-medical and medical face masks, which also incorporate air filters to provide enhanced protection.

Air filters are generally considered for an application based on their ability to filter out contaminants. At the most basic level, air filters are capable of blocking particles around 10 microns in size, which is representative of most dust and smoke particles. Better filters are able to block particles of a smaller size, down to 0.1 microns.

Another consideration for air filters is the blocking of bacterial and viral contaminants. While the particle size capabilities are one factor, it is also know to treat or coat the filter with a compound that has anti-bacterial and/or anti-viral properties in order to provide protection.

There are air filters known in the art, however, it remains desirable to have an air filter which provides an improved degree of protection against bacterial and viral contaminants and may be adaptable for multiple applications.

Accordingly, there remains a need for improvements in the art.

### SUMMARY OF THE INVENTION

In accordance with an aspect of the invention, there is provided to a four-layer air filter incorporating nanosilver in at least one interior layer.

According to an embodiment of the invention, there is provided an air filter, comprising: a first layer of a first material, the material comprising a non-woven polymer with a density of 25 grams per square meter (gsm); a second layer of a second material, the second material comprising a non-woven polymer with a density of 25 gsm coated with nanosilver; a third layer of a third material, the third material comprising a meltblown polymer with a density of 25 gsm; a fourth layer of a fourth material, the fourth material comprising a non-woven polymer with a density of 25 gsm and a plurality of embossed indentations thereon; wherein the layers are formed into the air filter by heat fusing the layers together and oriented such that external air flows through the first layer to the fourth layer when is use; and wherein the air filter provides non-medical anti-bacterial and anti-viral protection.

According to a further embodiment of the invention, the air filter provides a Particle Filtration Efficiency (PFE) of at least 98% and a Bacterial Filtration Efficiency (BFE) of at least 98%.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a cut-away side view of an air filter according to an embodiment;

FIG. 2 is a microscopic view of a first layer of non-woven polymer according to an embodiment;

FIG. 3 is a microscopic view of a second layer of non-woven polymer according to an embodiment;

FIG. 4 is a microscopic view of a third layer of meltblown polymer according to an embodiment;

FIG. 5 is a microscopic view of a fourth layer of non-woven polymer according to an embodiment.

Like reference numerals indicated like or corresponding elements in the drawings.

### DETAILED DESCRIPTION OF THE EMBODIMENTS

The present invention relates to an air filter and, in particular, to a multilayer air filer incorporating nanosilver as an anti-bacterial and/or anti-viral component.

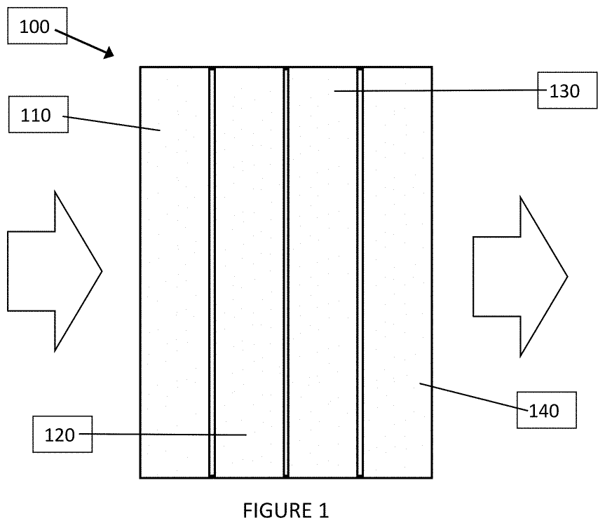

According to an embodiment as shown in FIG. 1, a multilayer air filter 100 is formed from four individual layers 110, 120, 130 and 140 of material fused together to form the air filter 100. Each layer is separately constructed and exhibits separate filtration properties. Air flow enters through the first layer 110 and exits through the fourth layer 140 as indicated by the arrows. In combination, the layers provide improved air filtration characteristics over a single layer filter.

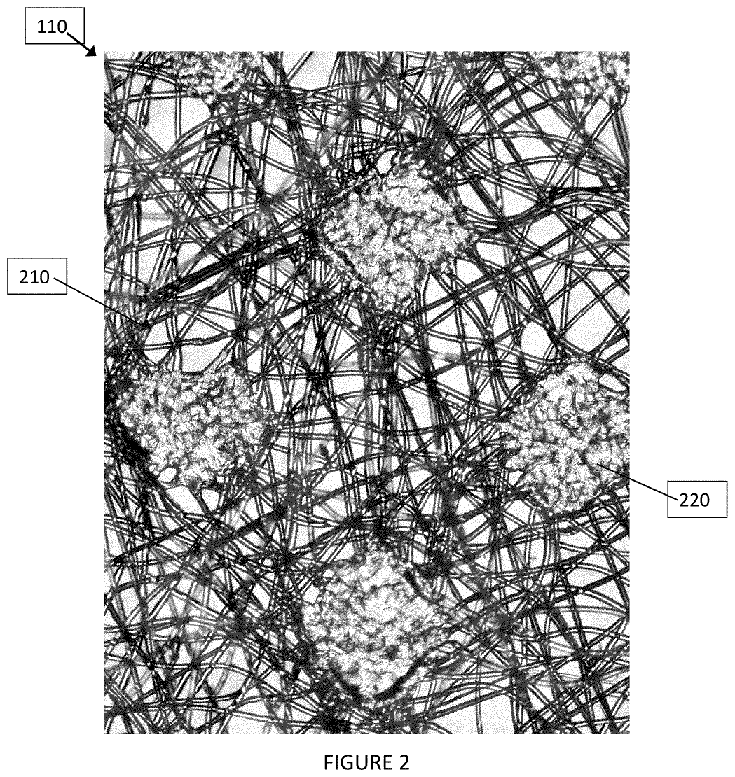

The first layer 110, as shown in FIG. 2, which is the outermost, incoming air-facing layer, is formed from a non-modified 25 gsm (grams/square meter) non-woven polymer 210 (e.g. polypropylene). While this first layer is non-modified, practical testing has revealed that particles 220 of nanosilver may transfer from the second layer 120 as discussed below and thus may be present upon microscopic examination of the first layer 110. The first layer 110 provides a degree of protection against micron-level particle and also provides protection to the second layer 120 from damage or nanosilver particle 220 removal due to contact.

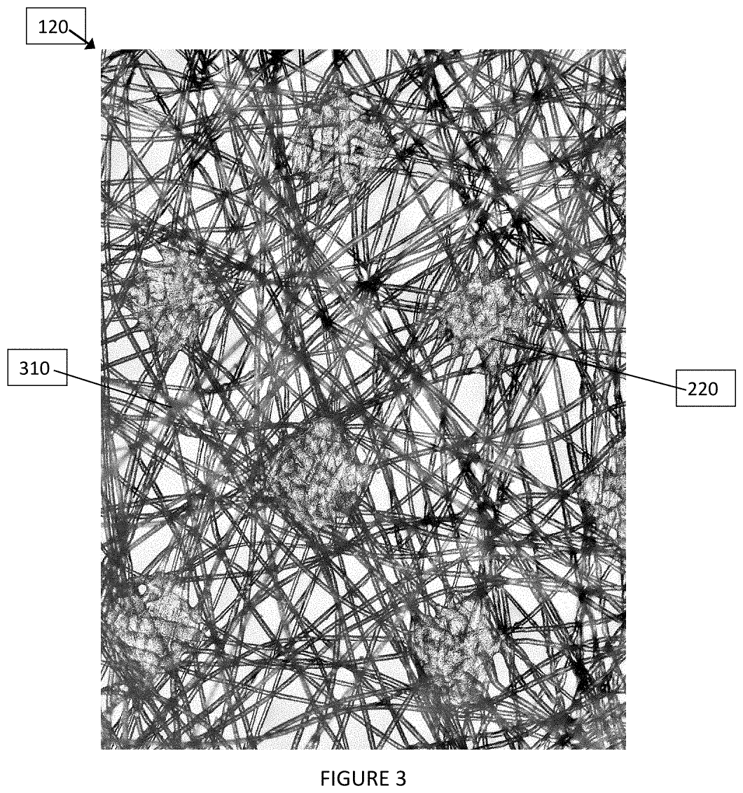

The second layer 120, as shown in FIG. 3, is also formed from a 25 gsm non-woven polymer 310 (e.g. polypropylene) which is coated with nanosilver particles 220. The nanosilver particles 220 provide anti-bacterial and anti-viral properties to the second layer 120. Testing has shown (see below) >98% bacterial filtration efficiency (BFE) and >98% particle filtration efficiency (PFE) for sub-micron particles when the second layer 120 is used in the air filter 100.

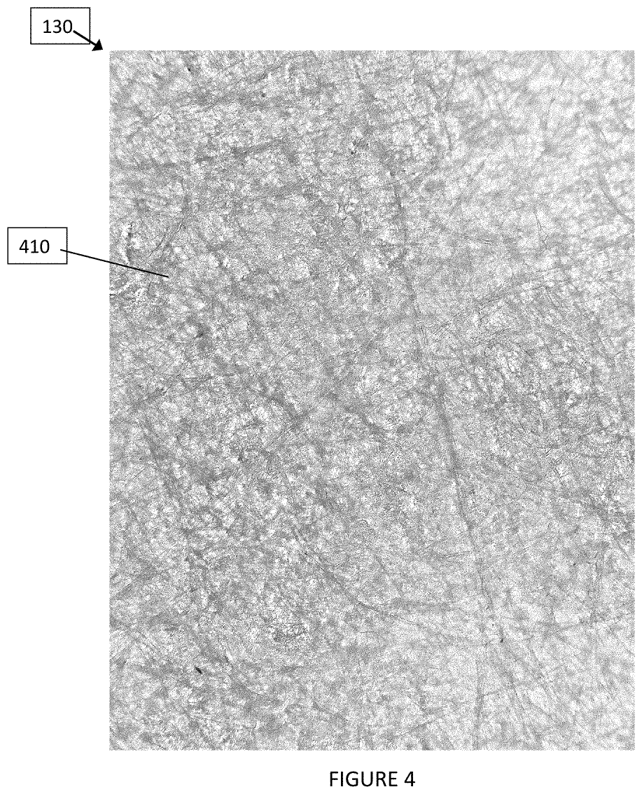

The third layer 130, as shown in FIG. 4, is formed from a 25 gsm meltblown polymer 410 such as polypropylene. The third layer 130 provides additional micron and sub-micron particle filtration.

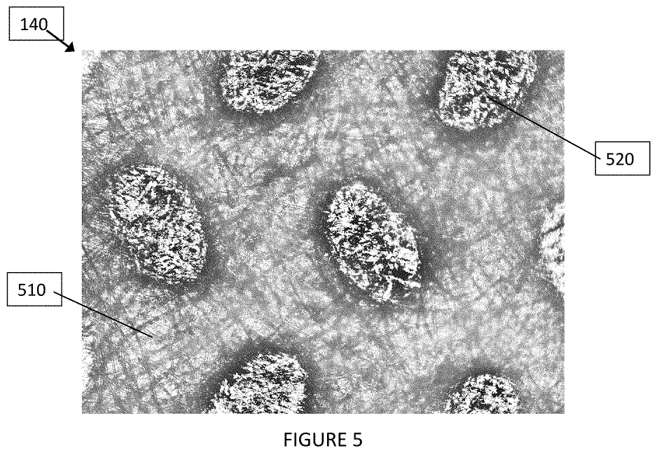

The fourth layer 140, as shown in FIG. 5, which is innermost relative to incoming air, is formed from a non-woven polymer 510 such as polypropylene, which is preferably comprised of an SMS non-woven polypropylene of three layers, a spunbond (or spunlaid) layer, a meltblown layer, and a second spunbond layer. The fourth layer 140 is further embossed with indentations 420 which act to trap moisture against the fourth layer 140. The ability to trap moisture is of particular use in applications such as face mask filters, where moisture from exhaled air may accumulate and interfere with breathing while the mask is in use for extended periods of time.

The four layers are each separately produced in the desired dimensions for the intended application (HVAC filter, face mask, CPAP, etc.). The four layers are then heat sealed together to form an air filter 100 as shown in FIG. 1. A coloring or marking may be added to either or both of the first layer 110 and fourth layer 140 in order to provide a visual indication of which layer is to be exposed to incoming air flow. The air filter 100 may further be cut or shaped during assembly as desired for the application e.g. air filters for use in cloth face masks may be tapered to better conform to the wearer's face.

Notably, it has been determined that nanosilver particles 220 may transfer from the second layer 120 to the first layer 110, either during manufacture or while in use. Accordingly, the nanosilver particles 220 will remain trapped within the air filter 100 and continue to maintain its anti-bacterial and anti-viral properties. This transfer may also provide a degree of protection against removal of the nanosilver particles 220 from the air filter 100 if the user (or another party) should touch or otherwise make contact with the first layer 110 of the air filter 100.

Fully assembled air filters 100 inserted into personal face masks and were tested for BFE and PFE as mentioned above. For the BFE testing, the ASTM F2101-19 Standard Test Method was performed using with a biological aerosol of Staphylococcus aureus. Testing results are shown in Table 1.

TABLE 1

| | Negative | Positive control | | | | | | Positive control |
|---|---|---|---|---|---|---|---|---|
| | control | before | Mask 1 | Mask 2 | Mask 3 | Mask 4 | Mask 5 | after |
| Plate No. | CFU/Plate | CFU/Plate | CFU/Plate | CFU/Plate | CFU/Plate | CFU/Plate | CFU/Plate | CFU/Plate |
| 1 | 0 | 175 | 4 | 6 | 7 | 5 | 6 | 180 |
| 2 | 0 | 160 | 2 | 3 | 4 | 3 | 4 | 167 |
| 3 | 0 | 148 | 1 | 2 | 2 | 1 | 4 | 152 |
| 4 | 0 | 133 | 1 | 1 | 1 | 0 | 2 | 146 |
| 5 | 0 | 120 | 0 | 0 | 0 | 1 | 1 | 131 |
| 6 | 0 | 107 | 0 | 1 | 1 | 0 | 0 | 123 |
| Total of | 0 | 843.00 | 8.00 | 13.00 | 15.00 | 10.00 | 17.00 | 899.00 |
| 6 plates | | | | | | | | |

Accordingly, the average BFE for the five tested masks was 98.55%.

Similarly, for PFE testing, an ASTM F2299/F2299M-03(2017) Standard Test Method was performed using latex spheres. Testing results are shown in Table 2.

| | Positive | Negative | Test | Actual | Percentage | Percentage |
|---|---|---|---|---|---|---|
| | Control | Control | Item | Count | Filtration | PFE |
| Mask No. | (A) | (B) | (C) | (D = C − B) | (E = D × 100/A) | (F = 100 − E) |
| 1 | 140000 | 812 | 1875 | 1063 | 7.592857143 | 92.40714286 |
| 2 | | | 787 | 0 | 0 | 100 |
| 3 | | | 1137 | 325 | 2.321428571 | 97.67857143 |
| 4 | | | 600 | 0 | 0 | 100 |
| 5 | | | 775 | 0 | 0 | 100 |

| | | |
|---|---|---|
| Average | 1.98 | 98.01 |

Accordingly, the average PFE for the five tested masks was 98.01%.

Therefore, standard testing data and results support the effectiveness of the four layer air filter 100 with a combination of polymer fabric for anti-particulate properties and nanosilver particles 220 for anti-bacterial and anti-viral properties.

As described herein, polypropylene is the preferred polymer for use in the layers, however other polymers may be used for one or more of the layers depending on the desired application and operating environment. Additionally, it is to be understood that the 25 gsm weight of the material is subject to standard manufacturing tolerances of +/−3 gsm.

As described herein, the air filter 100 is implemented as a filter for a personal non-medical face mask. However, the air filter 100 may be adapted, including via size and shape, for other applications, such as HVAC systems, vehicle air filters, and CPAP machines, for example.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. An air filter, comprising:
a first layer of a first material, the material comprising a non-woven polymer with a density of 25 grams per square meter (gsm);
a second layer of a second material, the second material comprising a non-woven polymer with a density of 25 gsm coated with nanosilver;
a third layer of a third material, the third material comprising a meltblown polymer with a density of 25 gsm;
a fourth layer of a fourth material, the fourth material comprising a non-woven polymer with a density of 25 gsm and a plurality of embossed indentations thereon;
wherein the layers are formed into the air filter by heat fusing the layers together and oriented such that external air flows through the first layer to the fourth layer when is use; and
wherein the air filter provides non-medical anti-bacterial and anti-viral protection.

2. The air filter of claim 1, wherein the non-woven polymer is polypropylene.

3. The air filter of claim 1, wherein the fourth material is a non-woven SMS (spunbond-meltblown-spunbond) material.

4. The air filter of claim 1, wherein the air filter is shaped to fit within a non-medical face mask.

5. The air filter of claim 1, wherein the air filter is shaped for use with a CPAP machine.

6. The air filter of claim 1, wherein the air filter provides a Particle Filtration Efficiency (PFE) of at least 98% and a Bacterial Filtration Efficiency (BFE) of at least 98%
